(19) United States
(12) Patent Application Publication
(10) Pub. No.: US 2021/0272684 A1
(43) Pub. Date: Sep. 2, 2021

(54) REDUCING REDUNDANT ALARMS

(71) Applicant: KONINKLIJKE PHILIPS N.V., ElNDHOVEN (NL)

(72) Inventors: Louis Nicolas ATALLAH, Boston, MA (US); Rohan JOSHI, Eindhoven (NL)

(21) Appl. No.: 17/258,830

(22) PCT Filed: Jul. 9, 2019
(86) PCT No.: PCT/EP2019/068391

§ 371 Date: Jan. 8, 2021

Related U.S. Application Data
(60) Provisional application No. 62/695,404, filed on Jul. 9, 2018.

(51) Int. Cl. G16H 40/40

(52) U.S. Cl. CPC G16H 40/40

(57) ABSTRACT

Methods and systems for reducing redundant alarms. The system may receive two or more different alarm sequences to infer relationships therebetween. Upon inferring that the datasets are coupled such that an alarm associated with one of the datasets follows an alarm associated with the other dataset, the system may suppress one of the alarms.

### TECHNICAL FIELD

Embodiments described herein generally relate to systems and methods for reducing alarms and, more particularly but not exclusively, to systems and methods for suppressing redundant alarms.

### BACKGROUND

In healthcare institutions such as hospitals or the like, many different medical devices are set up or otherwise configured differently and independently. Alarm-driven devices, such as those in healthcare environments, are generally configured to implement a “better safe than sorry” policy with a relatively high sensitivity and propensity to issue alarms.

However, an excess in alarms may lead to alarm fatigue in which clinicians develop a reduced sensitivity to monitor alarms and act upon them. This could have an adverse impact on the quality of care that a patient receives.

A need exists, therefore, for methods and systems that overcome the disadvantages of existing alarm-issuing techniques and devices.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

In one aspect, embodiments relate to a method for reducing redundant alarms. The method includes gathering a first set of data regarding a first entity using a first sensor device; gathering a second set of data regarding the first entity using a second sensor device; providing the first set of data and the second set of data to a processor executing instructions stored on a memory and providing a model; wherein the model is configured to infer a relationship between the first set of data and the second set of data, wherein the relationship indicates that an alarm associated with the second set of data is coupled with an alarm associated with the first set of data and suppress at least one of the coupled alarms; and storing the model for later usage so as to permit the stored model to be retrieved and used with a second entity sharing at least one characteristic with the first entity.

In some embodiments, the at least one characteristic includes at least one of age, gender, disease state, medication, interventions, and healthcare department.

In some embodiments, the method further includes adjusting an alarm threshold associated with at least one of the first set of data and the second set of data.

In some embodiments, the method further includes updating the model using later versions of the first and second sets of data.

In some embodiments, the model comprises at least one of a coupled hidden Markov model, a dynamic Bayesian network, and a recurrent neural network.

In some embodiments, the first entity is a patient, the first sensor device is a patient monitoring device, and the second sensor device is a ventilator.

In some embodiments, the model is further configured to infer the relationship based on at least one of time spent in at least one of a first alarm state associated with the first set of data and a second alarm state associated with the second set of data; and a time between a threshold breach and a generated alarm associated with at least one of the first set of data and the second set of data.

According to another aspect, embodiments relate to a system for reducing redundant alarms. The system includes an interface for receiving a first set of data regarding a first entity from a first sensor device and a second set of data regarding the first entity from a second sensor device; a processor executing instructions stored on a memory and providing a model, wherein the model is configured to at least infer a relationship between the first set of data and the second set of data, wherein the relationship indicates that an alarm associated with the second set of data is coupled with an alarm associated with the first set of data, and suppress at least one of the coupled alarms; and a database for storing the model for later usage so as to permit the stored model to be retrieved and used with a second entity sharing at least one characteristic with the first entity.

In some embodiments, the at least one characteristic includes at least one of age, gender, disease state, interventions, medication, and healthcare department.

In some embodiments, the processor is further configured to adjust an alarm threshold associated with at least one of the first set of data and the second set of data.

In some embodiments, the processor is configured to update the model using later versions of the first and second sets of data.

In some embodiments, the model comprises at least one of a coupled hidden Markov model, a dynamic Bayesian network, and a recurrent neural network.

In some embodiments, the first entity is a patient, and the first sensor device is a patient monitoring device and the second sensor device is a ventilator.

In some embodiments, the model is further configured to infer the relationship based on at least one of time spent in at least one of a first alarm state associated with the first set of data and a second alarm state associated with the second set of data, and a time between a threshold breach and a generated alarm associated with at least one of the first set of data and the second set of data.

According to another aspect, embodiments relate to a computer readable medium containing computer-executable instructions for a method for reducing redundant alarms. The medium includes computer-executable instructions for gathering a first set of data regarding a first entity using a first sensor device; computer-executable instructions for gathering a second set of data regarding the first entity using a second sensor device; computer-executable instructions for providing the first set of data and the second set of data to a processor executing instructions stored on a memory and providing a model, wherein the model is configured to infer a relationship between the first set of data and the second set of data, wherein the relationship indicates that an alarm associated with the second set of data is coupled with an alarm associated with the first set of data and suppress at least one of the coupled alarms; and computer-executable instructions for storing the model for later usage so as to permit the stored model to be retrieved and used with a second entity sharing at least one characteristic with the first entity.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a system for reducing redundant alarms in accordance with one embodiment;

FIG. 2 illustrates alarm sequence data for a coupled hidden Markov model in accordance with one embodiment;

FIG. 3 illustrates alarm sequence data considering time spent in each alarm state in accordance with one embodiment;

FIG. 4 illustrates a method of learning a relationship between existing alarm sequences and those of a new patient in accordance with one embodiment; and

FIG. 5 depicts a flowchart of a method for reducing redundant alarms in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

As discussed above, environments such as those in healthcare frequently rely on one or more monitoring devices to monitor a patient's health. These devices generally monitor one or more aspects of a patient's health and may issue an alarm upon detecting an anomaly or a possible cause for concern. In response to the issued alarm, a clinician may check on the patient and perform any required remedial action(s).

These alarms may originate from a plethora of medical devices that are each configured with their own characteristics. The majority of these devices operate independently from one another and are not coordinated with each other. Accordingly, multiple alarms relating to the same condition may be issued by different devices. This may contribute to the abundance of issued alarms, especially with devices that monitor a patient's cardiovascular system.

An excess of alarms may lead to alarm fatigue such that clinicians develop a reduced sensitivity to alarms and the motivation to act upon them. In fact, issues associated with alarms have been ranked as a top technology health hazard.

Furthermore, alarms are often accompanied by annoying sounds which can have a detrimental effect on patient comfort (e.g., by causing heightened stress, delirium, etc.). This is particularly true with vulnerable patients, such as infants or patients in intensive care units.

Clinicians or other medical staff are also often unable to accurately determine the device issuing an alarm. This is particularly true if the volume associated with the alarm is low. Similarly, it is common that clinicians ignore or silence alarms due to the large amount of alarms they hear in a typical work day. This inevitably increases the likelihood of missing critical alarms.

The systems and methods described herein may therefore learn or otherwise infer the relationship between alarm sequences in order to reduce redundant alarms. This may be done by connecting various machines together or by routing them to one machine. Or, in other embodiments, alarms may be managed by an external function or device that is connected to two or more machines.

A model may be trained and deployed and, when an event of connectivity is detected, issue only one alarm while suppressing one or more others. By learning the relationship between various devices and their associated alarm sequences, the systems and methods described herein may have more confidence regarding which alarms should be cancelled or otherwise suppressed. The model may be able to (1) learn the coupling between two or more sequences of alarms and then assess a new entity/new sequence of alarms; (2) learn the period of alarms for which the alarms are issued in each series (i.e., the time spent in each alarm state); and (3) after learning at least part of a series, compare the series to one or more previously-gathered series stored in a database to learn how the series can evolve over time.

In addition to patient monitoring devices, patients may also be connected to ventilators. Patient-ventilator interaction can be described as the relationship between two respiratory pumps. The first may involve the patient's pulmonary system, which is controlled by the patient's neuromuscular system, and the second may involve the ventilator, which is controlled by the ventilator settings and the function of the flow valve.

Since patient monitoring devices and ventilators interface with the patient in different ways, they may both detect the same patient deteriorations. For example, if there is a ventilator malfunction (e.g., a valve malfunction) and if the patient is not sufficiently ventilated, a ventilator alarm may be followed by a desaturation or a bradycardia alarm.

It is possible that, in this scenario, the ventilator alarm sounds before the patient's condition deteriorates to an extent that triggers an alarm from a separate patient monitoring device. This synergy between the patient monitoring devices and ventilators in detecting the same deteriorations can be exploited by the model and used to suppress redundant alarms and create early warnings for impending deteriorations.

Some alarms may be possible only in invasive ventilation (e.g., tube control) and vice versa. Moreover, the nature of ventilation captures information on the state of the patient. Thus, ventilator alarms and the type of ventilation used may both be inputs to the model.

Errors in device settings or device malfunction could also lead to false alarms. Knowledge regarding the relationships between alarms and the accompanying data may therefore be helpful to suppress false alarms as well.

For example, a SpO₂ sensor device may issue a desaturation alarm. However, data received from other devices may indicate that everything else is normal. The model, after having learned the relationship between alarm sequences for a particular patient, can therefore infer the desaturation alarm is an anomaly and that it is likely due to an equipment malfunction rather than a serious desaturation.

Although the present application largely discusses reducing redundant alarms in the context of healthcare and patient monitoring, it is contemplated that other applications may benefit from the features of the various embodiments described herein. For example, any type of electrical, mechanical, or computer-based systems that monitor separate types of data and issues alarms based on this data may benefit from the features described herein. These may include automobiles, aircraft, maritime vehicles, control systems, or the like. As another example, the systems and methods may monitor indicators related to financial markets or commodities and suppress any redundant alarms involved therewith.

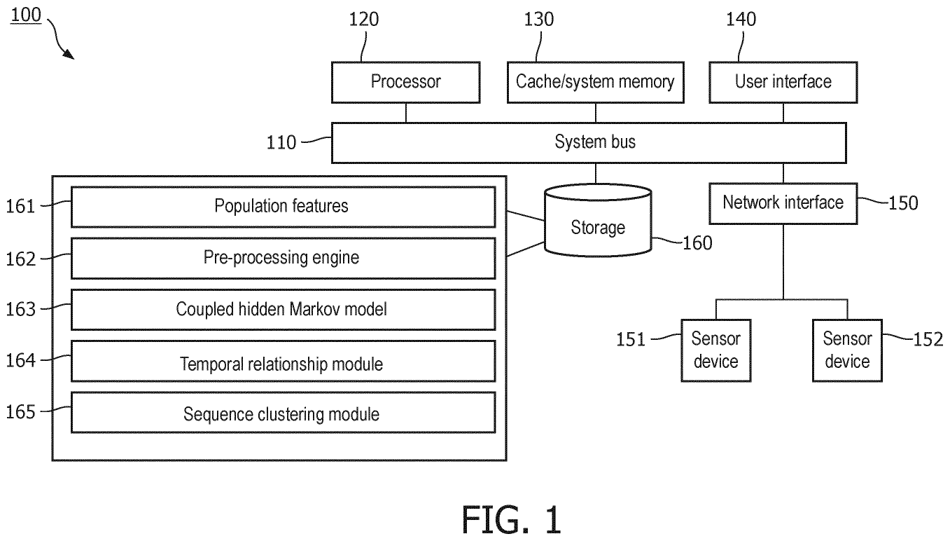

FIG. 1 illustrates a system 100 for reducing redundant alarms in accordance with one embodiment. As shown, the system 100 includes a processor 120, memory 130, a user interface 140, a network interface 150, and storage 160 interconnected via one or more system buses 110. It will be understood that FIG. 1 constitutes, in some respects, an abstraction and that the actual organization of the system 100 and the components thereof may differ from what is illustrated.

The processor 120 may be any hardware device capable of executing instructions stored on memory 130 or storage 160 or otherwise capable of processing data. As such, the processor 120 may include a microprocessor, field programmable gate array (FPGA), application-specific integrated circuit (ASIC), or other similar devices.

The memory 130 may include various memories such as, for example L1, L2, or L3 cache or system memory. As such, the memory 130 may include static random access memory (SRAM), dynamic RAM (DRAM), flash memory, read only memory (ROM), or other similar memory devices. The exact configuration of the memory 130 may vary as long as instructions for reducing redundant alarms can be executed.

The user interface 140 may include one or more devices for enabling communication with a user such as a clinician or other type of medical personnel. For example, the user interface 140 may include a display, a mouse, and a keyboard for receiving user commands. In some embodiments, the user interface 140 may include a command line interface or graphical user interface that may be presented to a remote terminal via the network interface 150.

The user interface 140 may execute on a user device such as a PC, laptop, tablet, mobile device, smartwatch, or the like. The exact configuration of the user interface 140 and the device on which it executes may vary as along as the features of various embodiments described herein may be accomplished.

The network interface 150 may include one or more devices for enabling communication with other hardware devices. For example, the network interface 150 may include a network interface card (NIC) configured to communicate according to the Ethernet protocol. Additionally, the network interface 150 may implement a TCP/IP stack for communication according to the TCP/IP protocols. Various alternative or additional hardware or configurations for the network interface 150 will be apparent.

The network interface 150 may be in operable communication with one or more sensor devices 151 and 152. In the healthcare context, these may include sensors configured as part of patient monitoring devices that gather various types of information regarding a patient's health. In this context, the sensors 151 and 152 may include SpO₂ sensors and ventilators, for example.

The type of sensors used may of course vary and depend on the entity and context. Accordingly, any type of sensor devices may be used as long as they can gather or otherwise obtain the required data regarding the entity under analysis. It is also noted that the model and any required processing may be embedded in any of the monitors and/or ventilators.

The various sensor devices used may be configured to generate alarms due to some deteriorating patient condition, for example. Oftentimes, different devices may each generate an alarm due to the same underlying event or patient condition. Accordingly, the system 100 may learn the relationship between alarm sequences of two or more devices such that when a first device generates an alarm, the system 100 may suppress additional alarms that otherwise would be generated by one or more other devices.

In other words, the system 100 may predict, based on an alarm received from a first device, that one or more other devices will subsequently generate an alarm. The system 100 may then suppress or ignore the subsequent alarms generated by the one or more other devices to prevent patients and/or clinicians from being subjected to an excess of alarms.

The sensor devices 151 and 152 may be in communication with the system 100 over one or more networks that may link the various components with various types of network connections. The network(s) may be comprised of, or may interface to, any one or more of the Internet, an intranet, a Personal Area Network (PAN), a Local Area Network (LAN), a Wide Area Network (WAN), a Metropolitan Area Network (MAN), a storage area network (SAN), a frame relay connection, an Advanced Intelligent Network (AIN) connection, a synchronous optical network (SONET) connection, a digital T1, T3, E1, or E3 line, a Digital Data Service (DDS) connection, a Digital Subscriber Line (DSL) connection, an Ethernet connection, an Integrated Services Digital Network (ISDN) line, a dial-up port such as a V.90, a V.34, or a V.34bis analog modem connection, a cable modem, an Asynchronous Transfer Mode (ATM) connection, a Fiber Distributed Data Interface (FDDI) connection, a Copper Distributed Data Interface (CDDI) connection, or an optical/DWDM network.

The network or networks may also comprise, include, or interface to any one or more of a Wireless Application Protocol (WAP) link, a Wi-Fi link, a microwave link, a General Packet Radio Service (GPRS) link, a Global System for Mobile Communication G(SM) link, a Code Division Multiple Access (CDMA) link, or a Time Division Multiple access (TDMA) link such as a cellular phone channel, a Global Positioning System (GPS) link, a cellular digital packet data (CDPD) link, a Research in Motion, Limited (RIM) duplex paging type device, a Bluetooth radio link, or an IEEE 802.11-based link.

The storage 160 may include one or more machine-readable storage media such as read-only memory (ROM), random-access memory (RAM), magnetic disk storage media, optical storage media, flash-memory devices, or similar storage media. In various embodiments, the storage 160 may store instructions for execution by the processor 120 or data upon which the processor 120 may operate.

For example, the storage 160 may include instructions for generating the model for detecting and monitoring relationships between two or more sets of time series data each associated with a device and/or sequences of alarms issued by two or more devices. The storage 160 may include population features 161, a pre-processing engine 162, a coupled hidden Markov model 163, a temporal relationship module 164, and a sequence clustering module 165.

The generated model may use a variety of techniques to learn or otherwise infer the relationship between alarm sequences of two or more sensor devices. The inferred relationships can be learnt for a particular patient and used to improve alarm management the patient and for future patients.

The model may consider population features 161 related to previously examined patients and associated data. The population features 161 may show the probability that one event (e.g., an alarm) will follow another event (e.g., another alarm) based on previously-collected data.

For example, a previous study conducted in a collaborating NICU based in the Netherlands analyzed alarms for 2,400 days. During this study, there were 355,000 critical alarms—18% of which were due to ventilators while the rest were from patient monitors. The five most prevalent alarms constituted 85% of all alarms. These alarms are summarized in Table 1 below:

TABLE 1
Alarm Summary

| Alarm | Device | % of all alarms | # of alarms |
|---|---|---|---|
| Desaturation | Patient monitor | 54.4% | 193,000 |
| Bradycardia | Patient monitor | 12.6% | 45,000 |
| Ppeak high | Ventilator | 8.1% | 29,000 |
| Patient loss | Ventilator | 6.3% | 22,500 |
| Tube control | Ventilator | 3.4% | 12,000 |

Based on this study, a number of relationships were identified. For example, 30% of all tube control alarms were followed by a desaturation alarm after a median time of 79 seconds, 35% of patient loss alarms were followed by a desaturation alarm after a median time of 2.2 minutes, 10% of all Ppeak high alarms were followed by a desaturation alarm after a median time of 3.2 minutes, and 7.5% of all tube control alarms were followed by a bradycardia alarm after a median time of 10 seconds. These types of relationships may be used to manage alarms associated with future patients.

The pre-processing engine 162 may receive data regarding an entity from at least sensor devices 151 and 152. As stated previously, this entity may be a patient in a healthcare setting. The pre-processing engine 162 may first perform any suitable pre-processing steps such as averaging, noise-reduction, or the like.

The coupled hidden Markov model 163 may consider previously gathered data related to the population at large (such as the population features 161) and, as data comes in for a particular patient, start to learn a more personalized version of the model for the patient. This allows the alarm sequence to adapt to the patient's particular physiology and their changing condition over time.

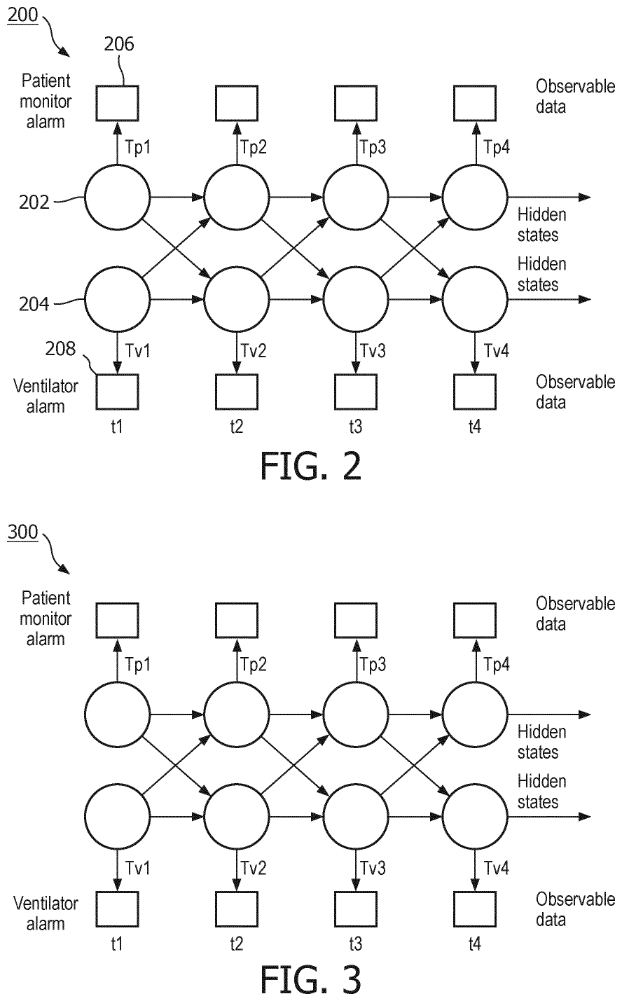

The coupled hidden Markov model 163 considers two different alarm series data sets, and learns the hidden states and the relationships between them. For example, FIG. 2 illustrates alarm sequence data 200 obtained from a patient monitoring device and a ventilator. The circles 202 and 204 represent hidden states related to data obtained by the patient monitoring device and the ventilator, respectively, at certain time intervals t. The squares 206 and 208 of FIG. 2 represent the observable states related to the data obtained by the patient monitoring device and the ventilator, respectively, at each time interval t. Accordingly, the model may learn the relationship between the two alarm sequences.

In addition to or in lieu of coupled hidden Markov models, the model may implement any suitable technique to learn the relationship between different alarm sequences. For example, the model may implement a dynamic Bayesian network, a recurrent neural network, or other neural networks to capture the relationship between alarm sequences over time. The exact implementation may of course vary as long as the features of the various embodiments described herein may be accomplished.

When an alarm is issued, it generally occurs for some duration of time until it is resolved. Alarms may be resolved automatically or only after some type of intervention by a clinician.

If a clinician is available and/or near a patient, the clinician may resolve the alarm relatively quickly (e.g., within 5-10 seconds). If there are no available clinicians in proximity to the patient, an issued alarm may occur for a longer duration of time until it is resolved. The duration of occurrence can be weighted and used in inferring relationships. For example, alarm states occurring for a longer duration of time may be considered more serious than alarm states that are resolved in a shorter period of time.

In some embodiments, therefore, the temporal relationship module 164 may consider the time spent in an alarm state. This data can help model the relationship between the different alarm sequence data.

For example, a coupled hidden Markov model may not only model the relationship between alarm sequences but also the time spent per state in each sequence. FIG. 3 illustrates alarm sequence data 300 from a patient monitoring device and a ventilator. However, the alarm sequence data 300 also includes time spent in T_{p,v} in each state for each device.

The sequence clustering module 165 may be applied when a new patient is connected to the sensor devices 151 and 152 for monitoring. The sequence clustering module 165 may access an online database of relationships and apply clustering methods to match the new patient with one or more similar data sets. For example, data associated with the new patient may be matched with existing data associated with previous patients based on one or more common characteristics between the patients. These characteristics may include, but are not limited to, patient age, gender, disease state, medication, interventions, and healthcare department.

The sequence clustering module 165 may not only match the new patient with previous patients based on some characteristic, but may also track how the alarm sequences for the previous patients developed over time. The sequence clustering module 165 may then use knowledge regarding this development to the new patient to help predict how the patient's alarm progression will evolve over time. Accordingly, alarm relationships can be adaptive and based on prior data.

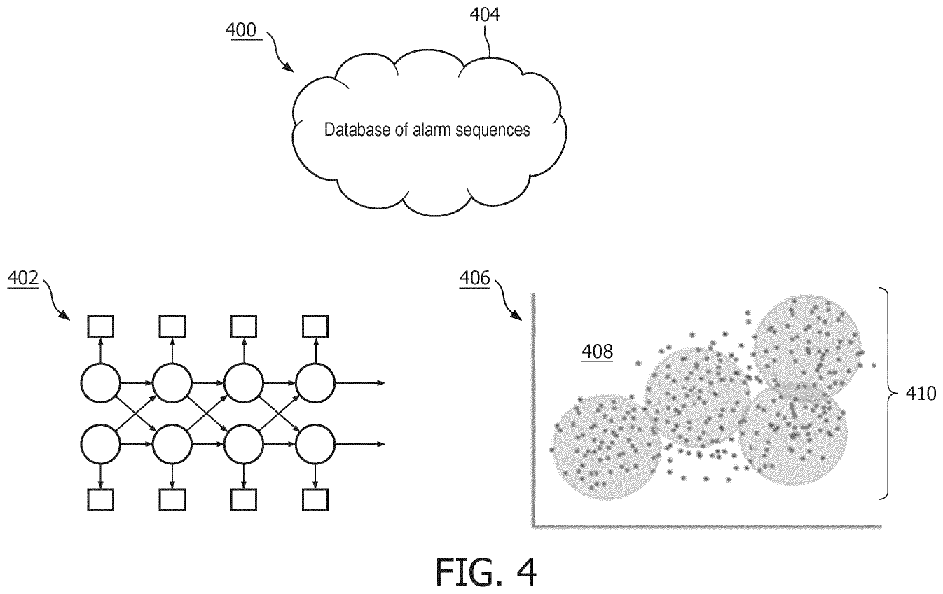

FIG. 4, for example, illustrates a method 400 of learning a relationship between existing alarm sequences and those of a new patient in accordance with one embodiment. Specifically, the model executed in method 400 may learn hidden parameters related to alarm sequences and reduce redundant alarms. Step 402 involves gathering alarm sequence data regarding the new patient. This alarm sequence data may be similar to the data of FIGS. 2 and 3, and include alarm data associated with a patient monitoring device and a ventilator.

In step 404, the alarm sequence data associated with the new patient may be compared to data stored in a database of alarm sequences (such as the population features 161 discussed above). This database may organize the alarm sequences based on one or more characteristics so that data associated with the new patient can be matched with data associated with similar patients. These characteristics may include patient age, gender, disease state, prescribed medication, interventions, healthcare institution department, or the like.

In step 406, the sequence clustering module 165 may cluster the alarms based on one or more characteristics of the patients to find one or more matches for the alarm sequence relationship. FIG. 4 illustrates a plot 408 showing a plurality of clusters 410 of alarm sequences. Each defined cluster 410 may include or otherwise be associated with a plurality of dots each representing an alarm sequence. An axis or the axes of plot 408 may each be labeled corresponding to a particular characteristic (e.g., age vs. disease state).

The new patient's characteristics may then be considered and compared to those on plot 408 to match the new patient with patients that share one or more common characteristics. The sequence clustering module 165 may assign the new patient to a cluster based on the one or more characteristics. Accordingly, the alarm sequence data of the new patient is associated with the alarm sequence data of one or more similar patients.

The sequence clustering module 165 may also consider the degree of similarity between the new patient and the similar patients. For example, the sequence clustering module 165 may execute a distance function with respect to the plot 408 so that data associated with previous patients who are, for example, closer in age to the new patient is weighed more heavily than data associated with other patients. The new patient's alarm sequence data (as well as knowledge of the inferred relationships) may be stored in the database of alarm sequences and used for further analyses.

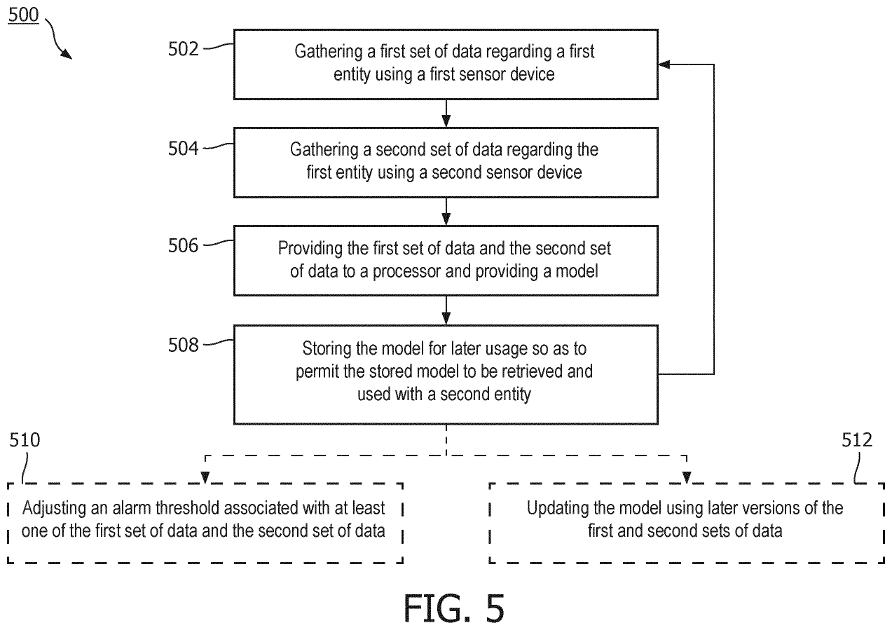

FIG. 5 depicts a flowchart of a method 500 for reducing redundant alarms in accordance with one embodiment. Step 502 involves gathering a first set of data regarding a first entity using a first sensor device. This first entity may be a patient being monitored in a healthcare institution, for example, and the first sensor device may be some type of patient monitoring device.

Step 504 involves gathering a second set of data regarding the first entity using a second sensor device. The second sensor device may be configured as or part of a ventilator, for example. The first and second sensor devices may be configured to gather data regarding the entity at predetermined time intervals to generate two time series data.

Step 506 involves providing the first set of data and the second set of data to a processor executing instructions stored on a memory and providing a model. The processor and memory may be similar to processor 120 and memory 130, respectively, of FIG. 1, for example.

The provided model may be configured to infer a relationship between the first set of data and the second set of data with respect to alarms. For example, and as discussed above, the relationship may indicate that an alarm associated with the second set of data is coupled with an alarm associated with the first set of data. Upon detecting this type of relationship, the model may then suppress one of the coupled alarms.

Accordingly, features of the systems and methods described herein may address the above-discussed problems associated with independently-configured alarm devices. That is, they may identify alarms that are coupled or otherwise related, even if issued by separate devices. The model may then suppress one of the alarms to prevent it from being issued. Clinicians (and/or patients) are therefore less likely to be bothered by an excess of alarms, many of which are unnecessary.

Step 508 involves storing the model for later usage so as to permit the stored model to be retrieved and used with a second entity sharing at least one characteristic with the first entity. Accordingly, data associated with new entities (e.g., new patients) may be compared with data associated with the first entity. As mentioned previously, knowledge regarding how alarm sequences progressed for one or more previous entities may be used to help predict the progression of alarm sequences for later entities.

Characteristics considered may include any one or more of age, gender, disease state, medication, interventions, and healthcare department. Accordingly, the data associated with the second entity is compared with data associated with one or more similar entities.

Step 510 is optional and involves adjusting an alarm threshold associated with at least one of the first set of data and the second set of data. The model may be updated for a given patient such that only data that exceeds a given threshold may issue an alarm. This may help avoid false positives that occur with only slight deviations from, for example, a patient's baseline data.

Step 512 is optional and involves updating the model using later versions of the first and second sets of data. As mentioned above, the progression of the alarm sequences may be monitored to continuously improve the model.

For example, data associated with the second sensor may not initially be coupled with the first set of data. However, as the patient's state evolves, data associated with the first and second sensors may become coupled with each other. This knowledge may therefore inform the model how the relationship between the first and second sets of data of later entities are at least likely to evolve to anticipate and suppress redundant alarms.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit ofthe disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

1. A method for reducing redundant alarms, the method comprising:
gathering a first set of data regarding a first entity using a first sensor device;
gathering a second set of data regarding the first entity using a second sensor device, wherein the second sensor device is different from the first sensor device;
providing the first set of data and the second set of data to a processor executing instructions stored on a memory and providing a model, wherein the model is configured to:
receive the first set of data and the second set of data;
infer a relationship between the first set of data and the second set of data, wherein the relationship indicates that an alarm associated with the second set of data is coupled with an alarm associated with the first set of data; and
suppress at least one of the coupled alarms; and
storing the model for later usage so as to permit the stored model to be retrieved and used with a second entity sharing at least one characteristic with the first entity.

2. The method of claim 1 wherein the at least one characteristic includes at least one of age, gender, disease state, medication, interventions, and healthcare department.

3. The method of claim 1 further comprising adjusting an alarm threshold associated with at least one of the first set of data and the second set of data.

4. The method of claim 1, further comprising updating the model using later versions of the first and second sets of data.

5. The method of claim 1 wherein the model comprises at least one of a coupled hidden Markov model, a dynamic Bayesian network, and a recurrent neural network.

6. The method of claim 1 wherein the first entity is a patient, and the first sensor device is a patient monitoring device and the second sensor device is a ventilator.

7. The method of claim 1 wherein the model is further configured to infer the relationship based on at least one of:
time spent in at least one of a first alarm state associated with the first set of data and a second alarm state associated with the second set of data, and
a time between a threshold breach and a generated alarm associated with at least one of the first set of data and the second set of data.

8. A system for reducing redundant alarms, the system comprising:
an interface for receiving:
a first set of data regarding a first entity from a first sensor device, and
a second set of data regarding the first entity from a second sensor device, wherein the second sensor device is different from the first sensor device;
a processor executing instructions stored on a memory and providing a model, wherein the model is configured to at least:
receive the first set of data and the second set of data;
infer a relationship between the first set of data and the second set of data, wherein the relationship indicates that an alarm associated with the second set of data is coupled with an alarm associated with the first set of data, and
suppress at least one of the coupled alarms; and
a database for storing the model for later usage so as to permit the stored model to be retrieved and used with a second entity sharing at least one characteristic with the first entity.

9. The system of claim 8 wherein the at least one characteristic includes at least one of age, gender, disease state, medication, interventions, and healthcare department.

10. The system of claim 8 wherein the processor is further configured to adjust an alarm threshold associated with at least one of the first set of data and the second set of data.

11. The system of claim 8 wherein the processor is configured to update the model using later versions of the first and second sets of data.

12. The system of claim 8 wherein the model comprises at least one of a coupled hidden Markov model, a dynamic Bayesian network, and a recurrent neural network.

13. The system of claim 8 wherein the first entity is a patient, and the first sensor device is a patient monitoring device and the second sensor device is a ventilator.

14. The system of claim 8 wherein the model is further configured to infer the relationship based on at least one of:
time spent in at least one of a first alarm state associated with the first set of data and a second alarm state associated with the second set of data, and
a time between a threshold breach and a generated alarm associated with at least one of the first set of data and the second set of data.

15. A non-transitory computer readable medium containing computer-executable instructions for a method for reducing redundant alarms, the medium comprising:
computer-executable instructions for gathering a first set of data regarding a first entity using a first sensor device;
computer-executable instructions for gathering a second set of data regarding the first entity using a second sensor device, wherein the second sensor device is different from the first sensor device;
computer-executable instructions for providing the first set of data and the second set of data to a processor executing instructions stored on a memory and providing a model, wherein the model is configured to:
receive the first set of data and the second set of data;
infer a relationship between the first set of data and the second set of data, wherein the relationship indicates that an alarm associated with the second set of data is coupled with an alarm associated with the first set of data, and
suppress at least one of the coupled alarms; and
computer-executable instructions for storing the model for later usage so as to permit the stored model to be retrieved and used with a second entity sharing at least one characteristic with the first entity.
